(19) United States
(12) Patent Application Publication
(10) Pub. No.: US 2008/0027103 A1
(43) Pub. Date: Jan. 31, 2008

(54) Novel Crystalline Forms 1

(75) Inventor: Carl-Gustav Sigfridsson, Molndal (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 11/622,596

(22) Filed: Jan. 12, 2007

(30) Foreign Application Priority Data
Jul. 4, 2006 (SE) PCT/SE06/00832

(51) Int. Cl. C07D 213/79; A61K 31/445

(52) U.S. Cl. 514/318; 546/194

(57) ABSTRACT

The present invention relates to 6-(4-{[(benzylsulfonyl)amino]carbonyl}piperidin-1-yl)-5-cyano-2-methylnicotinic acid isopropyl ester, form II, to a process for preparing such compound, to its utility as P2Y₁₂ inhibitor and as anti-thrombotic agent etc, its use as medicaments in cardiovascular diseases as well as pharmaceutical compositions containing it.

### FIELD OF THE INVENTION

The present invention provides novel crystalline forms of 6-(4-{[(benzylsulfonyl)amino]carbonyl}piperidin-1-yl)-5-cyano-2-methylnicotinic acid isopropyl ester, their use as medicaments, compositions containing them and processes for their preparation.

### BACKGROUND OF THE INVENTION

Platelet adhesion and aggregation are initiating events in arterial thrombosis. Although the process of platelet adhesion to the sub-endothelial surface may have an important role to play in the repair of damaged vessel walls, the platelet aggregation that this initiates can precipitate acute thrombotic occlusion of vital vascular beds, leading to events with high morbidity such as myocardial infarction and unstable angina. The success of interventions used to prevent or alleviate these conditions, such as thrombolysis and angioplasty is also compromised by platelet mediated occlusion or re-occlusion.

Haemostasis is controlled via a tight balance between platelet aggregation, coagulation and fibrinolysis. Thrombus formation under pathological conditions, like e.g. arteriosclerotic plaque rupture, is firstly initiated by platelet adhesion, activation and aggregation. This results not only in the formation of a platelet plug but also in the exposure of negatively charged phospholipids on the outer platelet membrane promoting blood coagulation. Inhibition of the build-up of the initial platelet plug would be expected to reduce thrombus formation and reduce the number of cardiovascular events as was demonstrated by the anti-thrombotic effect of e.g. Aspirin (BMJ 1994; 308: 81-106 Antiplatelet Trialists' Collaboration. Collaborative overview of randomised trials of antiplatelet therapy, I: Prevention of death, myocardial infarction, and stroke by prolonged antiplatelet therapy in various categories of patients.).

Platelet activation/aggregation can be induced by a variety of different agonists. However, distinct intracellular signalling pathways have to be activated to obtain full platelet aggregation, mediated via G-proteins G_{q}, G_{12/13} and Gᵢ (Platelets, A D Michelson ed., Elsevier Science 2002, ISBN 0-12-493951-1; 197-213: D Woulfe, et al. Signal transduction during the initiation extension, and perpetuation of platelet plug formation) In platelets, the G-protein coupled receptor P2Y₁₂ (previously also known as the platelet P_{2T}, P2T_{ac}, or P2Y_{cyc} receptor) signals via Gi, resulting in a lowering of intra-cellular cAMP and full aggregation (Nature 2001; 409: 202-207 G Hollopeter, et al. Identification of the platelet ADP receptor targeted by antithrombotic drugs.). Released ADP from dense-granules will positively feedback on the P2Y₁₂ receptor to allow full aggregation.

Clinical evidence for the key-role of the ADP-P2Y₁₂ feedback mechanism is provided by the clinical use of clopidogrel, an thienopyridine prodrug which active metabolite selectively and irreversibly binds to the P2Y₁₂ receptor, that has shown in several clinical trials to be effective in reducing the risk for cardiovascular events in patients at risk (Lancet 1996; 348: 1329-39: CAPRIE Steering committee, A randomised, blinded, trial of clopidogrel versus aspirin in patients at risk of ischaemic events (CAPRIE); N Engl J Med 2001; 345 (7): 494-502): The Clopidogrel in Unstable Angina to prevent Recurrent Events Trial Investigators. Effects of clopidogrel in addition to aspirin in patients with acute coronary syndromes without ST-segment elevation.). In these studies, the clinical benefit with a reduced bleeding risk as compared to thienopyridines (Sem Thromb Haemostas 2005; 31 (2): 195-204 J J J van Giezen & R G Humphries. Preclinical and clinical studies with selective reversible direct P2Y₁₂ antagonists.

Accordingly, it is an object of the present invention to provide potent, reversible and selective P2Y₁₂-antagonists as anti-thrombotic agents.

In the formulation of drug compositions, it is important for the drug substance to be in a form in which it can be conveniently handled and processed. This is of importance, not only from the point of view of obtaining a commercially viable manufacturing process, but also from the point of view of subsequent manufacture of pharmaceutical formulations comprising the active compound.

Further, in the manufacture of oral drug compositions, it is important that a reliable, reproducible and constant plasma concentration profile of drug is provided following administration to a patient.

Chemical stability, solid state stability, and “shelf life” of the active ingredients are also very important factors. The drug substance, and compositions containing it, should be capable of being effectively stored over appreciable periods of time, without exhibiting a significant change in the physico-chemical characteristics of the active component, e.g. its chemical composition, density, hygroscopicity and solubility.

Amorphous materials may present problems in this regard. For example, such materials are typically more difficult to handle and to formulate, provide for unreliable solubility, and are often found to be more unstable.

Thus, in the manufacture of commercially viable and pharmaceutically acceptable drug compositions, it is important, wherever possible, to provide the drug in a substantially crystalline and stable form(s).

### SUMMARY OF THE INVENTION

We have now surprisingly found that the crystalline forms of the invention are reversible and selective P2Y₁₂ antagonists. The compounds of the invention unexpectedly exhibit beneficial properties that render them particularly suitable for use in the treatment of diseases/conditions as described below. Examples of such beneficial properties are high potency, high selectivity, and an advantageous therapeutic window.

### DETAILED DESCRIPTION OF THE INVENTION

One aspect of the present invention is: 6-(4-{[(benzylsulfonyl)amino]carbonyl}piperidin-1-yl)-5-cyano-2-methylnicotinic acid isopropyl ester, form I in a first embodiment having the following XRPD peaks:

| Peak label | Angle (2-Theta, °) | Relative Intensity (%) |
|---|---|---|
| a | 4.945 | 100.0 |
| c | 9.337 | 26.8 |
| h | 14.327 | 60.9 |
| p | 20.674 | 20.2 |
| r | 21.865 | 25.6 |

In a 2^{nd} embodiment, 6-(4-{[(benzylsulfonyl)amino]carbonyl}piperidin-1-yl)-5-cyano-2-methylnicotinic acid isopropyl ester, form I, having the following XRPD peaks:

| Peak label | Angle (2-Theta, °) | Relative Intensity (%) |
|---|---|---|
| a | 4.945 | 100.0 |
| c | 9.337 | 26.8 |
| d | 9.863 | 15.9 |
| e | 10.324 | 18.1 |
| g | 13.398 | 17.6 |
| h | 14.327 | 60.9 |
| o | 20.130 | 15.1 |
| p | 20.674 | 20.2 |
| r | 21.865 | 25.6 |
| t | 23.033 | 18.4 |
| x | 24.789 | 17.9 |

In a 3^{rd} embodiment, 6-(4-{[(benzylsulfonyl)amino]carbonyl}piperidin-1-yl)-5-cyano-2-methylnicotinic acid isopropyl ester, form I, having the following XRPD peaks:

| Peak label | Angle (2-Theta, °) | Relative Intensity (%) |
|---|---|---|
| a | 4.945 | 100.0 |
| b | 7.142 | 3.4 |
| c | 9.337 | 26.8 |
| d | 9.863 | 15.9 |
| e | 10.324 | 18.1 |
| f | 12.960 | 2.9 |
| g | 13.398 | 17.6 |
| h | 14.327 | 60.9 |
| i | 14.800 | 4.1 |
| j | 17.096 | 4.6 |
| k | 18.344 | 6.5 |
| l | 18.668 | 5.0 |
| m | 19.094 | 9.6 |
| n | 19.800 | 5.2 |
| o | 20.130 | 15.1 |
| p | 20.674 | 20.2 |
| q | 21.073 | 7.3 |
| r | 21.865 | 25.6 |
| s | 22.563 | 7.8 |
| t | 23.033 | 18.4 |
| u | 23.849 | 13.7 |
| v | 24.209 | 6.4 |
| x | 24.789 | 17.9 |
| y | 25.458 | 6.1 |
| z | 26.055 | 5.0 |
| aa | 26.390 | 4.3 |
| ab | 27.072 | 6.3 |
| ac | 27.718 | 4.8 |
| ad | 28.721 | 9.8 |
| af | 29.783 | 10.5 |
| ag | 30.260 | 4.8 |
| ah | 30.840 | 4.8 |
| ai | 31.250 | 4.3 |
| ak | 34.511 | 4.3 |
| al | 36.014 | 4.4 |
| am | 37.174 | 4.1 |
| an | 37.855 | 3.5 |
| ao | 41.783 | 3.2 |
| ap | 43.320 | 4.9 |
| aq | 44.472 | 3.8 |
| ar | 45.346 | 3.1 |

Another aspect of the present invention is: 6-(4-{[(Benzylsulfonyl)amino]carbonyl}piperidin-1-yl)-5-cyano-2-methylnicotinic acid isopropyl ester, form II in a first embodiment having the following XRPD peaks:

| Peak label | Angle (2-Theta, °) | Relative Intensity (%) |
|---|---|---|
| A | 3.481 | 100.0 |
| B | 6.953 | 19.4 |
| C | 13.914 | 3.8 |
| E | 20.908 | 4.8 |

In a 2^{nd} embodiment, 6-(4-{[(benzylsulfonyl)amino]carbonyl}piperidin-1-yl)-5-cyano-2-methylnicotinic acid isopropyl ester, form II, having the following XRPD peaks:

| Peak label | Angle (2-Theta, °) | Relative Intensity (%) |
|---|---|---|
| A | 3.481 | 100.0 |
| B | 6.953 | 19.4 |
| C | 13.914 | 3.8 |
| D | 18.273 | 2.6 |
| E | 20.908 | 4.8 |
| F | 28.006 | 2.9 |

Another aspect of the present invention is a process for the preparation of the crystalline form I above comprising the steps of:

a) dissolving or suspending the compound 6-(4-{[(benzylsulfonyl)amino]carbonyl}piperidin-1-yl)-5-cyano-2-methylnicotinic acid isopropyl ester in isopropanol at ambient temperature or by refluxing;
b) allowing the material dissolved or suspended in the solution or suspension obtained in step in a) to crystallize, optionally during cooling to room temperature;
c) filtering the material obtained in step b) and isolating the crystalline product obtained.

A further aspect of the present invention is a process for the preparation of the crystalline form II above comprising the steps of:

a) dissolving or suspending the compound 6-(4-{[(benzylsulfonyl)amino]carbonyl}piperidin-1-yl)-5-cyano-2-methylnicotinic acid isopropyl ester in chloroform at ambient temperature or by refluxing;
b) allowing the material dissolved or suspended in the solution or suspension obtained in step in a) to crystallize, optionally during cooling to room temperature;
c) filtering the material obtained in step b) and isolating the crystalline product obtained.

Persons skilled in the art will appreciate that, in order to obtain compounds of the invention in an alternative and in some occasions, more convenient manner, the individual process steps mentioned hereinbefore may be performed in different order, and/or the individual reactions may be performed at different stage in the overall route (i.e. chemical transformations may be performed upon different intermediates to those associated hereinbefore with a particular reaction).

It will be appreciated that by those skilled in the art that the processes described above and hereinafter the functional groups of intermediate compounds may need to be protected by protecting groups.

Functional groups that it is desirable to protect include hydroxy, amino and carboxylic acid. Suitable protecting groups for hydroxy include optionally substituted and/or unsaturated alkyl groups (e.g. methyl, allyl, benzyl or tert-butyl), trialkyl silyl or diarylalkylsilyl groups (e.g. t-butyldimethylsilyl, t-butyldiphenylsilyl or trimethylsilyl) and tetrahydropyranyl. Suitable protecting groups for carboxylic acids include (C₁-C₆)alkyl or benzyl esters. Suitable protecting groups for amino include t-butyloxycarbonyl, benzyloxycarbonyl, 2-(trimethylsilyl)ethoxymethyl or 2-trimethylsilylethoxycarbonyl (Teoc).

The protection and deprotection of functional groups may take place before or after any reaction in the above mentioned processes.

Persons skilled in the art will appreciate that starting materials for any of the above processes can in some cases be commercially available.

Persons skilled in the art will appreciate that processes above could for some starting materials above be found in the general common knowledge.

The type of chemistry involved will dictate the need for protecting groups as well as sequence for accomplishing the synthesis.

The use of protecting groups is fully described in “Protective groups in Organic Chemistry”, edited by J W F McOmie, Plenum Press (1973), and “Protective Groups in Organic Synthesis”, 3^{rd} edition, T. W. Greene & P. G. M Wutz, Wiley-Interscince (1999).

All novel intermediates form a further aspect of the invention.

Here follows a definition of relative intensities:

| | | % relative intensity* |
|---|---|---|
| vs | very strong | >85% |
| s | strong | 27–85% |
| m | medium | 10–27% |
| w | weak | 5–10% |
| vw | very weak | <5% |

*the relative intensities are derived from diffractograms measured with variable slits.

It will be understood that the relative intensities of peaks may vary according to the orientation of the sample under test and on the type and setting of the instrument used so that the intensities in the X-ray powder diffraction traces included herein are illustrative and not intended to be used for absolute comparison.

### Pharmacological Data

Functional inhibition of the P2Y₁₂ receptor can be measured by in vitro assays using cell membranes from P2Y₁₂ transfected CHO-cells, the methodology is indicated below.

Functional inhibition of 2-Me-S-ADP induced P2Y₁₂ signalling: 5 μg of membranes were diluted in 200 μl of 200 mM NaCl, 1 mM MgCl₂, 50 mM HEPES (pH 7.4), 0.01% BSA, 30 μg/ml saponin and 10 μM GDP. To this was added an EC₈₀ concentration of agonist (2-methyl-thio-adenosine diphosphate), the required concentration of test compound and 0.1 μCi ³⁵S-GTPγS. The reaction was allowed to proceed at 30° C. for 45 min. Samples were then transferred on to GF/B filters using a cell harvester and washed with wash buffer (50 mM Tris (pH 7.4), 5 mM MgCl₂, 50 mM NaCl). Filters were then covered with scintilant and counted for the amount of ³⁵S-GTPγS retained by the filter. Maximum activity was that determined in the presence of the agonist and minimum activity in the absence of the agonist following subtraction of the value determined for nonspecific activity. The effect of compounds at various concentrations was plotted according to the equation

y=A+((B−A)/(1+((C/x)̂D)))

and IC₅₀ estimated where

### A is the bottom plateau of the curve i.e. the final minimum y value

### B is the top of the plateau of the curve i.e. the final maximum y value

C is the x value at the middle of the curve. This represents the log EC₅₀ value when A+B=100

### D is the slope factor.

x is the original known x values.

### Y is the original known y values.

The compound of the invention has an activity, when tested in the functional inhibition of 2-Me-S-ADPinduced P2Y₁₂ signalling assay described, at a concentration of 4 μM or below.

The compound of the invention act as P2Y₁₂ receptor antagonist and is therefore useful in therapy. Thus, according to a further aspect of the invention there is provided a compound of the invention for use in therapy.

In a further aspect there is provided the use of the compounds of the invention for the manufacture of a medicament for treatment of a platelet aggregation disorder. In another aspect of the invention there is provided the use of a compound of the invention for the manufacture of a medicament for the inhibition of the P2Y₁₂ receptor.

These compounds are useful in therapy, especially adjunctive therapy, particularly they are indicated for use as: inhibitors of platelet activation, aggregation and degranulation, promoters of platelet disaggregation, antithrombotic agents or in the treatment or prophylaxis of unstable angina, coronary angioplasty (PTCA), myocardial infarction, perithrombolysis, primary arterial thrombotic complications of atherosclerosis such as thrombotic or embolic stroke, transient ischaemic attacks, peripheral vascular disease, myocardial infarction with or without thrombolysis, arterial complications due to interventions in atherosclerotic disease such as angioplasty, endarterectomy, stent placement, coronary and other vascular graft surgery, thrombotic complications of surgical or mechanical damage such as tissue salvage following accidental or surgical trauma, reconstructive surgery including skin and muscle flaps, conditions with a diffuse thrombotic/platelet consumption component such as disseminated intravascular coagulation, thrombotic thrombocytopaenic purpura, haemolytic uraemic syndrome, thrombotic complications of septicaemia, adult respiratory distress syndrome, antiphospholipid syndrome, heparin-induced thrombocytopaenia and pre-eclampsia/eclampsia, or venous thrombosis such as deep vein thrombosis, venoocclusive disease, haematological conditions such as myeloproliferative disease, including thrombocythaemia, sickle cell disease; or in the prevention of mechanically-induced platelet activation in vivo, such as cardio-pulmonary bypass and extracorporeal membrane oxygenation (prevention of microthromboembolism), mechanically-induced platelet activation in vitro, such as use in the preservation of blood products, e.g. platelet concentrates, or shunt occlusion such as in renal dialysis and plasmapheresis, thrombosis secondary to vascular damage/inflammation such as vasculitis, arteritis, glomerulonephritis, inflammatory bowel disease and organ graft rejection, conditions such as migraine, Raynaud's phenomenon, conditions in which platelets can contribute to the underlying inflammatory disease process in the vascular wall such as atheromatous plaque formation/progression, stenosis/restenosis and in other inflammatory conditions such as asthma, in which platelets and platelet-derived factors are implicated in the immunological disease process.

According to the invention there is further provided the use of a compound according to the invention in the manufacture of a medicament for the treatment of the above disorders. In particular the compounds of the invention are useful for treating myocardial infarction, thrombotic stroke, transient ischaemic attacks, peripheral vascular disease and angina, especially unstable angina. The invention also provides a method of treatment of the above disorders which comprises administering to a patient suffering from such a disorder a therapeutically effective amount of a compound according to the invention.

In a further aspect the invention provides a pharmaceutical composition comprising a compound of the invention in combination with pharmaceutically acceptable adjuvants, diluents and/or carriers.

The compounds may be administered topically, e.g. to the lung and/or the airways, in the form of solutions, suspensions, HFA aerosols and dry powder formulations; or systemically, e.g. by oral administration in the form of tablets, pills, capsules, syrups, powders or granules, or by parenteral administration in the form of sterile parenteral solutions or suspensions, by subcutaneous administration, or by rectal administration in the form of suppositories or transdermally.

The compounds of the invention may be administered on their own or as a pharmaceutical composition comprising the compound of the invention in combination with a pharmaceutically acceptable diluent, adjuvant or carrier. Particularly preferred are compositions not containing material capable of causing an adverse, e.g. an allergic, reaction.

Dry powder formulations and pressurised HFA aerosols of the compounds of the invention may be administered by oral or nasal inhalation. For inhalation the compound is desirably finely divided. The compounds of the invention may also be administered by means of a dry powder inhaler. The inhaler may be a single or a multi dose inhaler, and may be a breath actuated dry powder inhaler.

One possibility is to mix the finely divided compound with a carrier substance, e.g. a mono-, di- or polysaccharide, a sugar alcohol or another polyol. Suitable carriers include sugars and starch. Alternatively the finely divided compound may be coated by another substance. The powder mixture may also be dispensed into hard gelatine capsules, each containing the desired dose of the active compound.

Another possibility is to process the finely divided powder into spheres, which break up during the inhalation procedure. This spheronized powder may be filled into the drug reservoir of a multidose inhaler, e.g. that known as the Turbuhaler® in which a dosing unit meters the desired dose which is then inhaled by the patient. With this system the active compound with or without a carrier substance is delivered to the patient.

The pharmaceutical composition comprising the compound of the invention may conveniently be tablets, pills, capsules, syrups, powders or granules for oral administration; sterile parenteral or subcutaneous solutions, suspensions for parenteral administration or suppositories for rectal administration.

For oral administration the active compound may be admixed with an adjuvant or a carrier, e.g. lactose, saccharose, sorbitol, mannitol, starches such as potato starch, corn starch or amylopectin, cellulose derivatives, a binder such as gelatine or polyvinylpyrrolidone, and a lubricant such as magnesium stearate, calcium stearate, polyethylene glycol, waxes, paraffin, and the like, and then compressed into tablets. If coated tablets are required, the cores, prepared as described above, may be coated with a concentrated sugar solution which may contain e.g. gum arabic, gelatine, talcum, titanium dioxide, and the like. Alternatively, the tablet may be coated with a suitable polymer dissolved either in a readily volatile organic solvent or an aqueous solvent.

For the preparation of soft gelatine capsules, the compound may be admixed with e.g. a vegetable oil or polyethylene glycol. Hard gelatine capsules may contain granules of the compound using either the above mentioned excipients for tablets, e.g. lactose, saccharose, sorbitol, mannitol, starches, cellulose derivatives or gelatine. Also liquid or semisolid formulations of the drug may be filled into hard gelatine capsules.

Liquid preparations for oral application may be in the form of syrups or suspensions, for example solutions containing the compound, the balance being sugar and a mixture of ethanol, water, glycerol and propylene glycol. Optionally such liquid preparations may contain colouring agents, flavouring agents, saccharine and carboxymethylcellulose as a thickening agent or other excipients known to those skilled in art.

The invention will be further illustrated with the following non-limiting examples:

### EXAMPLES

### General Experimental Procedure

Mass spectra was recorded on a Finnigan LCQ Duo ion trap mass spectrometer equipped with an electrospray interface (LC-ms) or LC-ms system consisting of a Waters ZQ using a LC-Agilent 1100 LC system.

1H NMR measurements were performed on a Varian Mercury VX 400 spectrometer, operating at a 1H frequency of 400 and Varian UNITY plus 400, 500 and 600 spectrometers, operating at 1H frequencies of 400, 500 and 600 respectively. Chemical shifts are given in ppm with the solvent as internal standard. Chromatography was performed using Biotage silica gel 40S, 40M, 12i or Merck silica gel 60 (0.063-0.200 mm). Flash chromatography was performed using either standard glass- or plastic-columns column or on a Biotage Horizon system. HPLC separations were performed on a Waters YMC-ODS AQS-3 120 Angstrom 3×500 mm or on a Waters Delta Prep Systems using Kromasil C8, 10 μm columns. Reactions performed in a microwave reactor were performed in a Personal Chemistry Smith Creator, Smith synthesizer or an Emrys Optimizer.

XRPD experiments were performed on a D8 Advance diffractometer (Bruxer AXS GmbH, Karlsruhe, Germany) with Bragg-Brentano geometry, equipped with a VÅNTEC-1 position sensitive detector (PSD). Nickel-filtered Cu K_{α} radiation was used. The samples, approx. 10 mg, were mounted on a zero-background holder (silicon crystal). Data were collected using continuous scan mode in the range 1-50° 2θ, with a step size of 0.017° and a step time of 0.5 sec. A variable (V20) divergence slit and a detector slit of 12 mm, corresponding to a 3.47° wide detector window, were applied.

### List of Used Abbreviations:

| Abbreviation | Explanation |
|---|---|
| br | Broad |
| BSA | Bovine Serum Albumine |
| d | Doublet |
| DCM | Dichloromethane |
| DIPEA | N,N-Diisopropylethylamine |
| DMF | Dimethylformamide |
| EDCI | N-[3-(dimethylamino)propyl]-N′-ethylcarbodiimide |
| | hydrochloride |
| EtOH | Ethanol |
| HOBt | 1-Hydroxybenzotriazole |
| Hz | Hertz |
| J | Coupling constant |
| m | Multiplet |
| MeOH | Methanol |
| MHz | Megahertz |
| mL | Millilitre |
| MS | Mass spectra |
| r.t. | room temperature |
| s | Singlet |
| THF | Tetrahydrofurane |

### Synthesis of 1-phenylmethanesulfonamide

1-phenylmethanesulfonyl chloride (0.75 mmol) was with a saturated solution of ammonia in MeOH (5 mL). After evaporation of the ammonia and MeOH the residues were dissolved in MeOH (5 mL) and to a few samples DMF (2 mL) was also added to dissolve the reaction mixtures. The solutions where then separately filtered through ISOLUTE SCX-2, (25 mL cartridge) containing acidic ion exchange resin (propylsulphonic acid type, 5 g). MeOH (16 mL) was used to rinse the product from the resin. After removal of the solvent the products were used without further purification as described in Method A below.

### Example 1

### 6-(4-{[(Benzylsulfonyl)amino]carbonyl}piperidin-1-yl)-5-cyano-2-methylnicotinic acid isopropyl ester

### (a) Isopropyl 2-((dimethylamino)methylene)-3-oxobutanoate

Isopropyl 3-oxobutanoate (200 mL, 1365 mmol) was stirred at r.t and dimethoxy-N,N-dimethylmethanamine (242 mL, 1706 mmol) was added drop-wise. The reaction mixture was allowed to stir at r.t overnight. The reaction mixture was concentrated under vacuum and then azeotroped with toluene (3×300 mL) and placed under high vacuum to afford isopropyl 2-((dimethylamino)methylene)-3-oxobutanoate as an oil, which was used without further purification. Yield: 272 g (100%).

¹H NMR (400 MHz, CDCl₃): δ 1.30 (6H, d, J=6.2 Hz), 2.32 (3H, s), 5.07-5.17 (1H, m), 7.64 (1H, s).

### (b) Isopropyl 5-cyano-2-methyl-6-oxo-1,6-dihydropyridine-3-carboxylate

NaH (33.359 g, 834.07 mmol) was suspended in THF (700 mL) and 2-cyanoacetamide (58.905 g, 700.62 mmol) added portion-wise at r.t. When gas evolution had stopped a solution of isopropyl 2-((dimethylamino)methylene)-3-oxobutanoate (147.72 g, 667.25 mmol) in THF (300 mL) was added and the system stirred at r.t overnight. The reaction mixture was concentrated under reduced pressure and the solids dissolved in the minimum amount of to hot water. 1N HCl was added to the solution until pH 1 and the solids isolated by filtration. The solids were dried under high vacuum to afford isopropyl 5-cyano-2-methyl-6-oxo-1,6-dihydropyridine-3-carboxylate as a solid, which was used without further purification. Yield: 123 g (84%).

¹H NMR (400 MHz, CDCl₃): δ 1.37 (6H, d, J=6.2 Hz), 2.84 (3H, s), 5.18-5.28 (1H, m), 8.50 (1H, s), 13.04 (1H, s).

MS ^{m}/z: 221 (M+1).

### (c) Isopropyl 6-chloro-5-cyano-2-methylnicotinate

Isopropyl 5-cyano-2-methyl-6-oxo-1,6-dihydropyridine-3-carboxylate (123.04 g, 558.70 mmol) was suspended in POCl3 (204.58 mL, 2234.8 mmol) and heated at 100° C. for 5 h. The reaction mixture was cooled to r.t and concentrated under reduced pressure. The residue was diluted with DCM and poured onto ice. The bi-phasic mixture was stirred at r.t and slowly quenched with solid K2CO3 until all the POCl3 had hydrolysed. The aqueous was extracted into DCM and the organics, dried (MgSO4) and passed through a silica plug. The organics were concentrated under reduced pressure to afford isopropyl 6-chloro-5-cyano-2-methylnicotinate as a solid, which was used without further purification. Yield: 106 g (79%).

¹H NMR (400 MHz, CDCl₃): δ 1.40 (6H, d, J=6.2 Hz), 2.90 (3H, s), 5.23-5.30 (1H, m), 7.26 (1H, s), 8.46 (1H, s).

MS ^{m}/z: 239 (M+1).

### (d) 1-(3-Cyano-5-(isopropoxycarbonyl)-6-methylpyridin-2-yl)piperidine 4-carboxylic acid

Isopropyl 6-chloro-5-cyano-2-methylnicotinate (25.000 g, 104.75 mmol), piperidine-4-carboxylic acid (14.205 g, 109.98 mmol) and DIPEA (d 0.742) (54.735 mL, 314.24 mmol) were suspended in EtOH (200 mL) and heated at reflux for 1 h. The reaction mixture was cooled to r.t and added drop-wise to KHSO4 (71.316 g, 523.74 mmol) in water (2000 mL). The solids were collected by filtration and dried under vacuum to afford 1-(3-cyano-5-(isopropoxycarbonyl)-6-methylpyridin-2-yl)piperidine-4-carboxylic acid as a solid, which was used without further purification. Yield: 35 g (100%).

1H NMR (400 MHz, CDCl₃): δ 1.35 (6H, d, J=6.2 Hz), 1.81-1.93 (2H, m), 2.04-2.12 (2H, m), 2.67-2.74 (4H, m), 3.26-3.36 (2H, m), 4.53-4.62 (2H, m), 5.15-5.23 (1H, m), 8.32 (1H, s).

MS ^{m}/z: 332 (M+1).

### (e) 6-(4-{[(Benzylsulfonyl)amino]carbonyl}piperidin-1-yl)-5-cyano-2-methylnicotinic acid isopropyl ester

1-[3-cyano-5-(isopropoxycarbonyl)-6-methylpyridin-2-yl]piperidine-4-carboxylic acid (30.00 g, 90.534 mmol), EDCI (26.03 g, 135.80 mmol), 1-phenylmethanesulfonamide (20.15 g, 117.69 mmol), HOBt (13.46 g, 99.59 mmol) and DIPEA (47.308 mL, 271.60 mmol) were suspended in DCM (400 mL) and stirred for 5 minutes until homogenous. Then the reaction mixture was refluxed for 4 h. The reaction mixture was cooled to r.t. and concentrated under reduced pressure. The crude reaction mixture was dissolved in EtOH (300 mL) and added drop-wise to a rapidly stirred solution of KHSO₄ (61.64 g, 452.67 mmol) in water (3000 mL). The product was collected by filtration, washed with water (3×400 mL) and dried under vacuum (44.00 g of dry product). The dry product was slurried in isopropyl alcohol (2000 mL) and stirred and heated at 50° C. for 2 h. The compound was isolated by filtration and dried under high vacuum to afford 6-(4-{[(benzylsulfonyl)amino]-carbonyl}piperidin-1-yl)-5-cyano-2-methylnicotinic acid Isopropyl ester as a solid. Yield: 37.41 g (85%).

¹H NMR (400 MHz, CDCl₃): δ 1.35 (6H, d, J=6.2 Hz), 1.74-1.90 (4H, m), 2.37-2.45 (1H, m), 2.73 (3H, s), 3.10-3.17 (2H, m), 4.63-4.67 (4H, m), 5.17-5.23 (1H, m), 7.33-7.42 (5H, m), 7.48 (1H, br s), 8.33 (1H, s).

MS ^{m}/z: 485 (M+1).

The crystalline form I obtained was characterised by the presence, in X-ray powder diffraction (XRPD) measurements, of peaks at about the 2-Theta and relative intensity values detailed in Table 1 below.

TABLE 1
XRPD Peaks for Form I of
6-(4-{[(benzylsulfonyl)amino]carbonyl}piperidin-1-yl)-
5-cyano-2-methylnicotinic acid isopropyl ester

| Peak label | Angle (2-Theta, °) | Relative Intensity (%) |
|---|---|---|
| a | 4.945 | 100.0 |
| b | 7.142 | 3.4 |
| c | 9.337 | 26.8 |
| d | 9.863 | 15.9 |
| e | 10.324 | 18.1 |
| f | 12.960 | 2.9 |
| g | 13.398 | 17.6 |
| h | 14.327 | 60.9 |
| i | 14.800 | 4.1 |
| j | 17.096 | 4.6 |
| k | 18.344 | 6.5 |
| l | 18.668 | 5.0 |
| m | 19.094 | 9.6 |
| n | 19.800 | 5.2 |
| o | 20.130 | 15.1 |
| p | 20.674 | 20.2 |
| q | 21.073 | 7.3 |
| r | 21.865 | 25.6 |
| s | 22.563 | 7.8 |
| t | 23.033 | 18.4 |
| u | 23.849 | 13.7 |
| v | 24.209 | 6.4 |
| x | 24.789 | 17.9 |
| y | 25.458 | 6.1 |
| z | 26.055 | 5.0 |
| aa | 26.390 | 4.3 |
| ab | 27.072 | 6.3 |
| ac | 27.718 | 4.8 |
| ad | 28.721 | 9.8 |
| af | 29.783 | 10.5 |
| ag | 30.260 | 4.8 |
| ah | 30.840 | 4.8 |
| ai | 31.250 | 4.3 |
| ak | 34.511 | 4.3 |
| al | 36.014 | 4.4 |
| am | 37.174 | 4.1 |
| an | 37.855 | 3.5 |
| ao | 41.783 | 3.2 |
| ap | 43.320 | 4.9 |
| aq | 44.472 | 3.8 |
| ar | 45.346 | 3.1 |

Form I of Example 1 was slurried in r.t. in chloroform. After 6 weeks in chloroform, Form I had been completely transformed into Form II. The melting point of Form I is approximately 210-212° C., whereas the melting point of Form II is approximately 204° C.

The crystalline form II obtained was characterised by the presence, in X-ray powder diffraction (XRPD) measurements, of peaks at about the 2-Theta and relative intensity values detailed in Table 2 below.

| Peak label | Angle (2-Theta, °) | Relative Intensity (%) |
|---|---|---|
| A | 3.481 | 100.0 |
| B | 6.953 | 19.4 |
| C | 13.914 | 3.8 |
| D | 18.273 | 2.6 |
| E | 20.908 | 4.8 |
| F | 28.006 | 2.9 |

1. 6-(4-{[(Benzylsulfonyl)amino]carbonyl}piperidin-1-yl)-5-cyano-2-methylnicotinic acid isopropyl ester, form II having the following XRPD peaks:

| Angle (2-Theta, °) | Relative Intensity (%) |
|---|---|
| 3.481 | 100.0 |
| 6.953 | 19.4 |
| 13.914 | 3.8 |
| 20.908 | 4.8 |

2. A crystalline form of the compound according to claim 1 having the following XRPD peaks:

| Angle (2-Theta, °) | Relative Intensity (%) |
|---|---|
| 3.481 | 100.0 |
| 6.953 | 19.4 |
| 13.914 | 3.8 |
| 18.273 | 2.6 |
| 20.908 | 4.8 |
| 28.006 | 2.9 |

3. A process for the preparing the compound of claim 1 or 2 comprising the steps of:
a) dissolving or suspending 6-(4-{[(benzylsulfonyl)amino]carbonyl}piperidin-1-yl)-5-cyano-2-methylnicotinic acid isopropyl ester in chloroform at ambient temperature or by refluxing;
b) allowing the compound dissolved or suspended in the solution or suspension obtained in step in a) to crystallize, optionally while cooling to room temperature;
c) filtering the suspension obtained in step b) and collecting the crystalline product obtained.

4-8. (canceled)

9. A pharmaceutical composition comprising 6-(4-{[(benzylsulfonyl)amino]carbonyl}piperidin-1-yl)-5-cyano-2-methylnicotinic acid isopropyl ester, form II having the following XRPD peaks:

| Angle (2-Theta, °) | Relative Intensity (%) |
|---|---|
| 3.481 | 100.0 |
| 6.953 | 19.4 |
| 13.914 | 3.8 |
| 20.908 | 4.8 |

or a crystalline form thereof having the following XRPD peaks:

| Angle (2-Theta, °) | Relative Intensity (%) |
|---|---|
| 3.481 | 100.0 |
| 6.953 | 19.4 |
| 13.914 | 3.8 |
| 18.273 | 2.6 |
| 20.908 | 4.8 |
| 28.006 | 2.9 |

in combination with at least one pharmaceutically acceptable adjuvant, diluent or carrier.

10. A method of treatment of a platelet aggregation disorder comprising administering to a patient suffering from such a disorder a therapeutically effective amount of 6-(4-{[(benzylsulfonyl)amino]carbonyl}piperidin-1-yl)-5cyano-2-methylnicotonic acid isopropyl ester, form II having the following XRPD peaks:

| Angle (2-Theta, °) | Relative Intensity (%) |
|---|---|
| 3.481 | 100.0 |
| 6.953 | 19.4 |
| 13.914 | 3.8 |
| 20.908 | 4.8 |

11. A method of treatment of a platelet aggregation disorder comprising administering to a patient suffering from such a disorder a therapeutically effective amount of a crystalline form of 6-(4-{[(benzylsulfonyl)amino]carbonyl}piperidin-1-yl)-5-cyano-2-methylnicotinic acid isopropyl ester, form II having the following XRPD peaks:

| Angle (2-Theta, °) | Relative Intensity (%) |
|---|---|
| 3.481 | 100.0 |
| 6.953 | 19.4 |
| 13.914 | 3.8 |
| 18.273 | 2.6 |
| 20.908 | 4.8 |
| 28.006 | 2.9 |
